(19) United States
(12) Patent Application Publication
(10) Pub. No.: US 2022/0354862 A1
(43) Pub. Date: Nov. 10, 2022

(54) LIPOSOME DELIVERY OF PSYCHEDELICS

(71) Applicant: Mind Medicine, Inc., New York, NY (US)

(72) Inventors: Robert BARROW, Madison, WI (US); Gennady N. SMAGIN, Newark, DE (US)

(73) Assignee: Mind Medicine, Inc., New York, NY (US)

(21) Appl. No.: 17/733,389

(22) Filed: Apr. 29, 2022

Related U.S. Application Data
(60) Provisional application No. 63/184,000, filed on May 4, 2021.

(51) Int. Cl. A61K 31/55; A61K 9/127; A61K 31/4745; A61K 31/675; A61K 31/4045; A61K 31/137; A61K 9/00

(52) U.S. Cl. CPC A61K 31/55; A61K 9/127; A61K 31/4745; A61K 31/675; A61K 31/4045; A61K 31/137; A61K 9/0019

(57) ABSTRACT

A composition of a psychedelic in a liposome formulation, wherein the composition provides preferential distribution of the psychedelic at the CNS and blood, and reduced distribution at peripheral organs. A method of treating a patient, by administering a composition of a psychedelic in a liposome formulation to the patient, and preferentially distributing the psychedelic at the CNS with a reduced exposure in target peripheral organs (e.g., heart).

### BACKGROUND OF THE INVENTION

### 1. Technical Field

The present invention relates to compositions and methods for delivering psychedelics with liposomes. More specifically, the present invention relates to compositions and methods for delivering ibogaine and noribogaine with liposomes.

### 2. Background Art

Ibogaine is an indole alkaloid that is a psychoactive substance found naturally in plants such as Tabernanthe iboga, Voacanga africana, and Tabernaemontana undulate. West African indigenous peoples have used ibogaine in ritual, ordeal, or initiation potions in large doses and as a stimulant in small doses. Ibogaine provides a visionary phase and introspection phase after administration.

Ibogaine has been studied as a treatment for opioid addiction. For example, U.S. Pat. No. 4,499,096 to Lotsof discloses administering ibogaine, ibogaine HCl or other salts of ibogaine to a heroin addict. A single treatment was effective for about 6 months, and a series of 4 treatments was effective for approximately 3 years. The treatments consisted of the oral administration of ibogaine or its salts in dosage ranges of 6 mg/kg to 19 mg/kg. The minimum effective dose was 400 mgs and dosage increase above 1000 mgs were found to be unnecessary. Treatments were effective in 71% of the cases. U.S. Pat. No. 4,587,243 to Lotsof discloses administration to a cocaine or amphetamine abuser, U.S. Pat. No. 4,857,523 to Lotsof discloses administration to an alcohol dependent, and U.S. Pat. No. 5,026,697 to Lotsof discloses administration to a nicotine or tobacco addict.

Noribogaine is a metabolite of ibogaine and can also be used in treatments for drug addiction and pain. For example, U.S. Pat. No. 7,220,737 to Mash discloses a method of alleviating pain in a patient by administering systemically noribogaine at a therapeutically effective dosage, and a method for treating drug addiction (involving drug dependency or drug abuse) during withdrawal therapy by administering noribogaine to a patient at a dosage sufficient to reduce or eliminate one or more symptoms associated with withdrawal.

However, clinical studies of ibogaine stopped for many years due to side effects of cardiotoxicity and individuals who have used ibogaine have died from these effects. High doses of ibogaine and noribogaine cause long QT syndrome by blocking hERG potassium channels in the heart (Koenig, et al., Molecules, 2015 February; 20(2): 2208-2228).

Liposomes are drug delivery vehicles that are used for stabilizing compounds, aiding in cellular and tissue uptake, and improving biodistribution of compounds. Generally, a liposome is a spherical vesicle with an aqueous solution core having at least one lipid bilayer as a hydrophobic membrane. Hydrophobic molecules can be inserted into the bilayer membrane and hydrophilic molecules can be surrounded in the aqueous core.

There are several types of liposomes used for delivery: conventional liposomes, sterically-stabilized liposomes, ligand-targeted liposomes, and combinations thereof (Sercombe, et al. Front Pharmacol. 2015; 6: 286). Conventional liposomes include the lipid bilayer of phospholipids and cholesterol enclosing the aqueous core and have been used to reduce toxicity of compounds but have the drawback of rapid elimination from the bloodstream. Sterically-stabilized liposomes were developed to improve stability and blood circulation times by using polyethylene glycol (PEG) as the lipid bilayer. However, sterically-stabilized liposomes can have a reduced ability to interact with intended targets. Ligand-targeted liposomes can be used to deliver drugs to specific cell types or organs. Ligands can include antibodies, peptides or proteins, and carbohydrates. Monoclonal antibodies can provide stability and high binding avidity due to two binding sites on the molecule. Surface-coupled ligands also have high motional freedom to position themselves for optimal interactions with substrates due to the dynamic properties of the liposome structure. Combinations of liposome types can be created to take advantage of the properties of each type.

Liposomes can be used to deliver drugs to the brain with passive or active targeted delivery. Fasudil is approved for cerebral vasospasm but can also be used potentially for stroke. Clinical trials for stroke were stopped because of difficulty in free drug penetrating the blood brain barrier. After encapsulating in PEG-liposomes (a passive delivery), diffusion and accumulation in the I/R region was shown and there was suppressed volume of damaged brain tissue in rats (Fukuta T, et al. Neuroprotection against cerebral ischemia/reperfusion injury by intravenous administration of liposomal fasudil. International Journal of Pharmaceutics. 2016; 506(1-2):129-137). Active targeted delivery can also be used. Liposomal doxorubicin is an anti-cancer drug, and glutathione PEGylated liposomal doxorubicin has been shown to have a higher brain concentration of doxorubicin than a passive liposomal PEG formulation of doxorubicin (Gaillard P J, et al. Pharmacokinetics, brain delivery, and efficacy in brain tumor-bearing mice of glutathione pegylated liposomal doxorubicin (2B3-101). PLoS One. 2014; 9(1):e82331).

Therefore, there remains a need for safe administration of ibogaine and noribogaine at all dosage levels by increasing blood and CNS exposure while limiting tissue distribution in target organs (e.g., heart) sensitive to toxic effects.

### SUMMARY OF THE INVENTION

The present invention provides for a composition of a psychedelic in a liposome formulation, wherein the composition provides preferential distribution of the psychedelic at the CNS and blood and reduced distribution in peripheral organs, e.g., heart.

The present invention provides for a method of treating a patient, by administering a composition of a psychedelic in a liposome formulation to the patient, and preferentially distributing the psychedelic at the CNS.

### DESCRIPTION OF THE DRAWINGS

Other advantages of the present invention are readily appreciated as the same becomes better understood by reference to the following detailed description when considered in connection with the accompanying drawings wherein:

FIG. 1 is a graph of concentration of noribogaine in plasma (ng/ml) of rats injected intravenously (IV) with noribogaine and BTLS (brain-targeted liposomes)-noribogaine injected by the same route.

### DETAILED DESCRIPTION OF THE INVENTION

The present invention provides for a composition of a psychedelic in a liposome formulation. The composition can be used in methods of preferentially distributing psychedelics at the CNS. The composition provides increased CNS efficacy with increased distribution to the CNS and blood for a given dose with reduced distribution and binding to peripheral organs and target organs for toxicity (e.g., heart).

The psychedelics in the present invention can be, but are not limited to, ibogaine, noribogaine, lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, solvates thereof, tartrates thereof, analogs thereof, or homologues thereof. Preferably, the dose of the psychedelic is one that provides a meaningful effect. A dose of 1-1000 mg of ibogaine or noribogaine can be used. A dose of 0.01-1 mg (10-1000 μg) can be used of LSD. Psilocybin can be dosed at 5-50 mg, psilocin can be dosed at 1-100 mg, mescaline can be dosed at 50-800 mg, 5-MeO-DMT can be dosed at 1-20 mg, DMT can be dosed at 20-100 mg, DOI can be dosed at 0.1-5 mg, and DOB can be dosed at 0.1-5 mg. Effects of the psychedelic drug can last 1-12 hours after administration, and the individual can be supervised by medical personnel such as a psychiatrist during this time. If lower doses are given, medical supervision can be unnecessary.

The liposomes can generally be of the type of conventional liposomes, sterically-stabilized liposomes, ligand-targeted liposomes, or combinations thereof. The liposomes can be designed to preferentially target the CNS for distribution of the psychedelic into the CNS as opposed to systemic distribution, which contributes to the cardiotoxicity of compounds like ibogaine and noribogaine or other toxic effects related to peripheral organs.

Passive or active targeting with liposomes can be used to deliver drugs (i.e., the psychedelics) across the blood brain barrier (Rita Nieto Montesinos (Oct. 25, 2017). Liposomal Drug Delivery to the Central Nervous System, Liposomes, Angel Catala, IntechOpen, DOI: 10.5772/intechopen.70055). With passive targeting, use of PEG or derivatives, poly(vinyl pyrrolidone) (PVP), or poly(acryl amide) (PAA) in the lipid bilayer can prevent the recognition of liposomes by opsonins and reduces clearance by the reticuloendothelial system (RES) to provide enhanced permeability and retention (EPR) effects. Active targeting uses small-molecule ligands, peptides, aptamers, monoclonal antibodies (mAbs) or fragments on the surface of the liposome. The liposome can then target the brain after recognizing biochemical transport systems expressed at brain endothelial cells such as adsorptive-mediated endocytosis (AME), carrier-mediated transport (CMT), and receptor-mediated endocytosis (RME). AME relies on an electrostatic interaction between a positively charged moiety and the negatively charged sites on the luminal surface of plasma membrane and brain capillaries. Since AME occurs in other organs, this method can be less specific for the brain. The CMT systems are localized at the brain capillary endothelium and mediate the passage of small molecular weight nutrients across the BBB. This includes transporters for D-glucose (GLUT1), large neutral amino acid (LAT1), small neutral amino acids (EAAT), cationic amino acids (CAT1), monocarboxylic acids (MCT1), and organic cations (OCT). Therefore, liposomes can include GLUT1, LAT1, EAAT, CAT1, MCT1, OCT, and combinations thereof on their surface to target the CNS. RME systems require the binding of a ligand to a specific receptor located on the luminal membrane of the BBB, and the receptor-ligand binding induces the internalization of receptor-ligand complexes within an endocytic vesicle. Liposomes can include ligands such as low-density lipoproteins (LDL), low-density lipoprotein receptor-related protein 1 (LRP-1), insulin, insulin-like growth factors (IGF-I, IGF-II), interleukin-1 (IL-1), folic acid (FA) and transferrin (Tf), or mAbs or their fragments (Fab′, F(ab′)₂) to use RME systems. One particularly useful mAb is OX26 that targets brain capillary endothelial cells.

Liposomes allow for precise active drug distribution to a specific site in the body. So, depending on the effect needed, specific patients can receive liposomes loaded with different concentrations of drug. This allows for higher, otherwise more toxic concentrations to be locally delivered without otherwise expected toxicity. Specifically with respect to noribogaine or ibogaine, the use of liposomes allows for therapeutic doses to be administered that reduce the side effects of hERG inhibition, cardiotoxicity, and/or the development of cardiac arrhythmias by reducing effective concentrations (distribution) and exposure in the heart tissue.

The compounds of the present invention are administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compounds of the present invention can be administered in various ways. It should be noted that they can be administered as the compound and can be administered alone or as an active ingredient in combination with pharmaceutically acceptable carriers, diluents, adjuvants, and vehicles. The compounds can be administered orally, transcutaneously, subcutaneously or parenterally including intravenous, intramuscular, and intranasal administration. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention.

The doses can be single doses or multiple doses or a continuous dose over a period of several hours, days, weeks, or months.

When administering the compound of the present invention orally, it will generally be formulated in an immediate release capsule, immediate release tablet, modified release capsule or tablet (including enteric coatings), solution, or suspension. When administering the compound of the present invention parenterally, it will generally be formulated in a sublingual or buccal orally dissolving tablet, dissolving film, intranasal powder, intranasal solution, inhaled powder, inhaled solution, transdermal patch, transdermal patch with microneedles or other permeation enhancers, or as a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The present invention provides for a method of treating a patient, by administering a composition of a psychedelic in a liposome formulation to the patient, and preferentially distributing the psychedelic at the CNS and blood and reducing distribution to target peripheral organs such as the heart. The liposome formulation allows for the psychedelic to cross the blood brain barrier and be distributed to the brain and CNS, instead of just systemically. The composition can also stay bound to a receptor in the CNS long enough to provide a beneficial effect. This provides a safer delivery of psychedelics such as ibogaine and noribogaine. The liposome formulation can include any modification described above (i.e., passive, or active targeting) to direct the liposomes to the CNS and cross the blood brain barrier. The psychedelics can be administered in a dose as described above.

The condition or disease being treated can include, but is not limited to, anxiety disorders (including anxiety in advanced stage illness e.g. cancer, as well as generalized anxiety disorder), depression (including post partum depression, major depressive disorder and treatment-resistant depression), headache disorder (including cluster headaches and migraine headache), obsessive compulsive disorder (OCD), personality disorders (including conduct disorder), stress disorders (including adjustment disorders and post-traumatic stress disorder), drug disorders (including alcohol dependence or withdrawal, nicotine dependence or withdrawal, opioid dependence or withdrawal, cocaine dependence or withdrawal, methamphetamine dependence or withdrawal), other addictions (including gambling disorder, eating disorder, and body dysmorphic disorder), pain, neurodegenerative disorders (such as dementia, Alzheimer's Disease, Parkinson's Disease), autism spectrum disorder, eating disorders, or neurological disorders (such as stroke).

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Pharmacokinetic Evaluation of the Tissue Distribution of Two Test Articles (Noribogaine and BTLS (Brain-Targeted Liposomes)-Noribogaine) in Rats

This study was conducted in adult male Sprague Dawley rats, weighing 250-275 g on arrival. Animals were housed according to the vivarium SOP on a 12-hr light cycle with food and water ad libitum.

TABLE 1 shows test items, TABLE 2 shows reference items, and TABLE 3 shows animal information.

TABLE 1
Test items

| | |
|---|---|
| Name | BTLS-Noribogaine for intravenous |
| | injection |
| Therapeutic Area/Clinical | Substance abuse, opioid use |
| indication | disorder or alcohol use disorder |
| Characteristics & Physical State | Liquid |
| Storage conditions during delivery | On Ice |
| Storage conditions after delivery | 2-8° C. |
| Appearance | White solution (as empty |
| | liposomal solution) |
| | Transparency: no (opaque) |
| | Turbidity: high |
| | Odor: odorless |
| Manufacturing date | December 2021 |
| Expiry Date (stability at storage | March 2022 |
| condition) | |
| pH | 5.5-7.5 |
| Osmolality | 270-320 Osm/kg H2O |
| Protected from light | Yes, |
| | During dosing, the Test Item will be |
| | wrapped in aluminum foil |

TABLE 2
Reference items

| | |
|---|---|
| Name: | IV Injection Noribogaine |
| Characteristics & Physical | Liquid |
| State: | |
| Storage conditions during | on Ice |
| delivery: | |
| Storage conditions after | 2-8° C. |
| delivery: | |
| Type of container/s: | Falcon tubes |
| Number of container/s: | 8 |
| | ~9 ml per tube |
| Concentration: | 0.5 mg/ml |
| Appearance: | Turbidity: Yellowish clear solution |
| | Transparency: not turbid (fluid) formula- |
| | tion, homogeneous without precipitates. |
| | Odor: |
| Manufacturing date: | December 2021 |
| Expiry Date | March 2022 |
| CoA or SDS to be provided | No |
| with material | |
| Surface Coating: | No |
| pH | 5.5-7.5 |
| Osmolality | 270-320 Osm/kg H2O |
| Protected from light | During dosing, the Test Item will be |
| | wrapped in aluminum foil |

TABLE 3
Animal information

| | |
|---|---|
| Animals: | Sprague-Dawley rats, ♂, 7 weeks of age at study |
| | initiation, about 220-250 g. |
| Supplier: | Envigo RMS (Israel) LTD (ISO 9001:2015 Certificate |
| | No.: GB06/68708) |
| Total number | n = 72 |
| of animals: | |
| Identification & | Animals were given a unique identification ear |
| Randomization: | number. During acclimation period animals were |
| | randomly assigned to the various test groups. |
| Weight | Should not exceed ±20% of the mean weight at the |
| variation: | time of treatment initiation. Mean group weight should |
| | be similar as much as possible among groups, at study |
| | initiation. The heaviest animals will be subjected for |
| | the first day of dosing. |

TABLE 4 shows the constitution of test groups.

TABLE 4

| | | | | | Treatment | | | | Scheduled Termination | | | | | |
|---|---|---|---|---|---|---|---|---|---|---|---|---|---|---|
| | | | staggering¹ | | | | Dose | Dose | (post-dosing) | | | | | |
| | Group | Sub- | Day | Day | Test | | Level | Volume | 5 | 15 | 30 | 1 | 2 | 8 |
| Gp. | Size | Gp. | 0 | 2 | Material | Route | (mg/kg) | (ml/kg) | min | min | min | hr | hr | hr |
| 1M | n = 36 | a | n = 3 | n = 3 | BTLS- | Intravenous | 2.5 | 5 | ✓ | | | | | |
| | | b | n = 3 | n = 3 | Noribogaine | (IV) | | | | ✓ | | | | |
| | | c | n = 3 | n = 3 | (Test Item) | | | | | | ✓ | | | |
| | | d | n = 3 | n = 3 | | | | | | | | ✓ | | |
| | | e | n = 3 | n = 3 | | | | | | | | | ✓ | |
| | | f | n = 3 | n = 3 | | | | | | | | | | ✓ |
| 2M | n = 36 | a | n = 3 | n = 3 | IV Injection | | 2.5 | 5 | ✓ | | | | | |
| | | b | n = 3 | n = 3 | Noribogaine | | | | | ✓ | | | | |
| | | c | n = 3 | n = 3 | (Reference | | | | | | ✓ | | | |
| | | d | n = 3 | n = 3 | Item) | | | | | | | ✓ | | |
| | | e | n = 3 | n = 3 | | | | | | | | | ✓ | |
| | | f | n = 3 | n = 3 | | | | | | | | | | ✓ |

The tested material is a metabolite (noribogaine) of naturally occurring product, ibogaine. Noribogaine is a medication for treatment of psychiatric disorders, including opioid use disorder, depression, and PTSD

BTLS-Noribogaine is an experimental formulation. The study used liposomes to encapsulate the drug substance for targeted delivery. The application of liposomes to assist drug delivery has already had a major impact on many biomedical areas. They have been shown to be beneficial for stabilizing therapeutic compounds, overcoming obstacles to cellular and tissue uptake, and improving biodistribution of compounds to target sites in vivo. This enables effective delivery of encapsulated compounds to target sites while minimizing systemic toxicity. Liposomes are defined as phospholipid vesicles consisting of one or more concentric lipid bilayers enclosing discrete aqueous spaces.

72 rats were divided into 12 groups of 6. Test articles were dosed at 10 mg/kg of active substance (generic and liposomal formulation) and administered IV through the tail vein as a bolus. Animals were sampled for 24 hours after administration. Terminal blood and tissue samples (brain, heart, kidney, liver) were collected at 0.08, 0.25, 1, 2, 4 and 8 hours after the dose. The study compared the two groups, noribogaine and BTLS-noribogaine formulation. Concentration of noribogaine in the plasma and tissues was measured ex vivo and pharmacokinetic analysis was performed. The PK analysis demonstrated basic parameters, such as peak concentration, peak time, half-life of elimination, and clearance.

Results

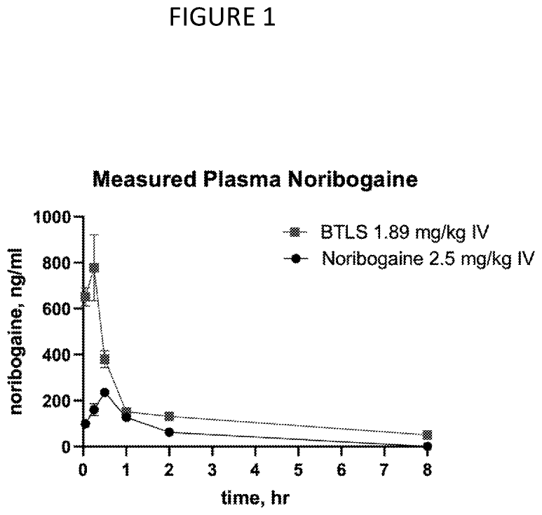

FIG. 1 shows the measured concentration of noribogaine in plasma (ng/ml) of rats injected IV with noribogaine (2.5 mg/kg) and BTLS-noribogaine (1.89 mg/kg), as indicated in TABLE 4. N=5-6/group.

TABLE 5 shows calculated pharmacokinetic parameters after bolus IV administration of Noribogaine and BTLS-noribogaine.

TABLE 5

| Variable | BTLS | SEM | NOR | SEM |
|---|---|---|---|---|
| T_half, hr | 0.7987 | 0.0692 | 1.4558 | 0.1385 |
| Tmax, hr | 0.1933 | 0.0358 | 0.5 | 0 |
| Cmax, ng/ml | 816.1667 | 125.5437 | 223.6 | 14.2077 |
| AUC 0-t, | 593.5433 | 64.462 | 272.626 | 26.6708 |
| ng/ml*hr\hat{ }2 | | | | |
| Vd, mL | 0.0031 | 0.0013 | 0.0132 | 0.0024 |
| Cl, ml/hr | 0.0026 | 2.0E−4 | 0.0064 | 6.0E−4 |
| Vss, mL | 0.0032 | 5.0E−4 | 0.0119 | 0.0012 |

Table key:
T_half, time required for plasma concentration of a drug to decrease by 50%;
Tmax-time for the substance to reach the maximum concentration;
Cmax-maximum concentration,
Vd-volume of distribution;
AUC 0-t-area under the curve until the last measurable concentration. AUC is approximated by a series of trapezoids;
Cl clearance;
Vss-steady state volume of distribution.

Administration of BTLS-noribogaine compared to noribogaine significantly changed pharmacokinetic parameters in rats. Maximum plasma concentration (Cₘₐₓ) was higher (360%) than in animals administered with noribogaine. Area under the curve (AUC) was >200% higher than in animals administered with noribogaine, demonstrating increased plasma exposure with comparable dose. Moreover, volume of distribution (V_{d} and Vₛₛ) for BTLS-noribogaine is significantly lower than for noribogaine, demonstrating lower plasma and tissue binding of the compound.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition comprising a psychedelic in a liposome formulation, wherein said composition provides preferential distribution of the psychedelic at the CNS and blood, and reduced distribution at peripheral organs.

2. The composition of claim 1, wherein said psychedelic is chosen from the group consisting of ibogaine, noribogaine, lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, solvates thereof, tartrates thereof, analogs thereof, and homologues thereof.

3. The composition of claim 1, wherein said liposome formulation is made from liposomes chosen from the group consisting of conventional liposomes, sterically-stabilized liposomes, ligand-targeted liposomes, and combinations thereof.

4. The composition of claim 1, wherein said liposome formulation is designed to preferentially target the CNS and provide a low concentration in peripheral organs.

5. The composition of claim 4, wherein said liposome formulation includes a mechanism to deliver the psychedelic across the blood brain barrier chosen from the group consisting of passive targeting and active targeting.

6. The composition of claim 1, wherein said psychedelic is chosen from the group consisting of ibogaine and noribogaine, and said composition reduces side effects of hERG inhibition, cardiotoxicity, and the development of cardiac arrhythmias by reducing distribution and exposure at the heart.

7. A method of treating a patient, including the steps of:
administering a composition of a psychedelic in a liposome formulation to the patient; and
preferentially distributing the psychedelic at the CNS and blood and reducing distribution to target peripheral organs.

8. The method of claim 7, wherein the psychedelic is chosen from the group consisting of ibogaine, noribogaine, lysergic acid diethylamide (LSD), psilocybin, psilocin, mescaline, 5-methoxy-N,N-dimethyltryptamine (5-MeO-DMT), dimethyltryptamine (DMT), 2,5-dimethoxy-4-iodoamphetamine (DOI), 2,5-dimethoxy-4-bromoamphetamie (DOB), salts thereof, solvates thereof, tartrates thereof, analogs thereof, and homologues thereof.

9. The method of claim 7, wherein the liposome formulation is made from liposomes chosen from the group consisting of conventional liposomes, sterically-stabilized liposomes, ligand-targeted liposomes, and combinations thereof.

10. The method of claim 7, wherein said preferentially distributing step is accomplished by the liposome formulation.

11. The method of claim 10, wherein the liposome formulation includes a mechanism to deliver the psychedelic across the blood brain barrier chosen from the group consisting of passive targeting and active targeting.

12. The method of claim 7, wherein the composition stays bound at a receptor in the CNS long enough to provide a beneficial effect.

13. The method of claim 7, wherein the psychedelic is chosen from the group consisting of ibogaine and noribogaine, and further including the steps of reducing side effects of hERG inhibition, cardiotoxicity, and the development of cardiac arrhythmias by reducing distribution and exposure at the heart.

14. The method of claim 7, wherein the patient has a condition or disease chosen from the group consisting of anxiety disorders, depression, headache disorder, obsessive compulsive disorder (OCD), personality disorders, stress disorders, drug disorders, gambling disorder, eating disorder, body dysmorphic disorder, pain, neurodegenerative disorders, autism spectrum disorder, eating disorders, and neurological disorders.
